(19) United States
(12) Patent Application Publication
(10) Pub. No.: US 2006/0253102 A1
(43) Pub. Date: Nov. 9, 2006

(54) Non-expandable transluminal access sheath

(76) Inventors: Edward J. Nance, Corona, CA (US); Jay Lenker, Laguna Beach, CA (US); Onnik Tchulluian, Carlsbad, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/313,400

(22) Filed: Dec. 21, 2005

Related U.S. Application Data
(60) Provisional application No. 60/637,906, filed on Dec. 21, 2004.

(51) Int. Cl. A61M 25/00; A61M 5/178

(52) U.S. Cl. 604/525; 604/164.13; 604/170.02

(57) ABSTRACT

A transluminal sheath is disclosed that permits instrumentation to be passed therethrough. The transluminal sheath comprises a composite structure with an inner layer, an outer layer, and a reinforcing layer. The materials comprising the inner and outer layer are plastically deformable and maintain their shape, once bent into a specific configuration. The reinforcing layer further has radiopacity enhancing coatings to improve visibility under fluoroscopy and a system of flutes running longitudinally, to enhance fluid transport and reduce friction.

### PRIORITY INFORMATION

This application claims priority to U.S. provisional application Ser. No. 60/637,906, filed on Dec. 21, 2004, titled Non-Expandable Transluminal Sheath, the entirety of which is hereby incorporated herein by reference.

### BACKGROUND OF THE INVENTION

1. Field of the Invention

The present invention relates to medical devices and, more particularly, to methods and devices for accessing a mammalian body lumen or cavity.

2. Description of the Related Art

A wide variety of diagnostic or therapeutic procedures involve the introduction of an access device through a natural access pathway. The access device provides an access lumen, which is used to introduce into the patient diagnostic or therapeutic instrumentation. A general objective of such access devices is to minimize the cross-sectional area of the access lumen while maximizing the available space for the diagnostic or therapeutic instrumentation.

One environment where access devices are used for the urinary tract of the human or other mammal. The urinary tract is relatively short natural lumen that is substantially free from the severe tortuosity found in many endovascular applications. Ureteroscopy is an example of one type of therapeutic interventional procedure that is used in the urinary tract. Ureteroscopy is a minimally invasive procedure that provides access to the upper urinary tract (i.e. the ureter). Access to the ureter is made via the urethra, another body lumen, and the bladder, which is a body cavity. Ureteroscopy is for stone extraction, stricture treatment, or stent placement.

Often, to perform a procedure in the ureter, a cystoscope is placed into the bladder through the urethra. A guidewire is next placed, through the working channel of the cystoscope and under direct visual guidance, into the target ureter. Once guidewire control is established, the cystoscope is removed and the guidewire is left in place. A ureteral sheath or catheter is next advanced through the urethra over the guidewire, through the bladder and on into the ureter. The guidewire may now be removed to permit instrumentation of the ureteral sheath or catheter. In a variation on the procedure, the guidewire may be left in place during instrumentation. In yet another variation on the procedure, an additional, or “safety”, guidewire is inserted into the urinary system.

Often, current techniques involve advancing a flexible, 10 to 18 French, ureteral catheter with integral flexible, tapered obturator, sometimes called a dilator, over the guidewire. Because axial pressure is required to advance and place each catheter, care must be taken to avoid kinking the tapered obturator during advancement so as not to compromise the working lumen of the catheter through which instrumentation, such as ureteroscopes and stone extractors, must now be placed. Furthermore, operators must avoid advancing devices, sheaths, catheters, and instrumentation, against strictures or tortuous ureteral walls with high forces that could cause injury to the ureteral wall or kidney.

One of the issues that arise during ureteroscopy is the presence of an obstruction or stenosis in the ureter, which is sometimes called a stricture, that prohibits a catheter with a large enough working channel from being able to be advanced into the ureter. Such conditions may preclude the minimally invasive approach and require more invasive surgical procedures in order to complete the task. Urologists may be required to use catheters with suboptimal central lumen size because they are the largest catheters that can be advanced to the proximal end of the ureter. Alternatively, urologists may start with a larger catheter and then need to downsize to a smaller catheter, a technique that results in a waste of time and expenditure. Finally, a urologist may need to dilate the ureter with a dilation system, such as a bougie or balloon dilatation catheter, before placing the current devices. In most cases, it is necessary for the urologist to perform fluoroscopic evaluation of the ureter to determine the presence or absence of strictures and what size catheter would work for a given patient.

Additional information regarding ureteroscopy can be found in Su, L, and Sosa, R. E., Ureteroscopy and Retrograde Ureteral Access, Campbell's Urology, 8th ed, vol. 4, pp. 3306-3319 (2002), Chapter 97. Philadelphia, Saunders. Another reference is Moran, M. E., editor, Advances in Ureteroscopy, Urologic Clinics of North America, vol. 31, No. 1 (February 2004), the entirety of which are hereby expressly incorporated by reference herein.

A need therefore remains for improved access technology, which allows a device to be transluminally passed through a relatively small diameter duct, such as is in the urinary tract, while accommodating the introduction of relatively large diameter instruments. In certain applications, a sheath or catheter would enter a vessel or body lumen with a diameter of about 8 to 18 French, and be able to pass instruments through a central lumen, which is maximized for the application. Furthermore, the sheath or catheter would desirably have improved flexibility and trackability over guidewires relative to currently available devices. The sheath or catheter would advantageously be visible under fluoroscopy and would be relatively inexpensive to manufacture. Furthermore, the sheath or catheter would be kink resistant and minimize abrasion and damage to instrumentation being passed therethrough. The catheter or sheath should also minimize the risk of injury to adjacent anatomic structures. Such injury could result in bleeding, development of subsequent strictures, or leakage of urine into surrounding-renal structures.

### SUMMARY OF THE INVENTION

Accordingly, one embodiment of the present invention comprises a transluminal sheath adapted for insertion into a mammalian body vessel or cavity. The sheath comprises an axially elongate composite sheath tube with a proximal and a distal end and a central through lumen. The composite sheath tube comprises an outer layer, an inner layer, and a reinforcing layer wherein the outer layer and the inner layer are fabricated from polymeric materials. A hub is affixed to the proximal end of the sheath tube. A central obturator is configured to occlude the central lumen of the sheath during insertion. A guidewire lumen extends within the obturator.

Another embodiment of the invention comprises an access sheath configured provide access to the ureter, kidney, or bladder. In an embodiment, the sheath would have an introduction outside diameter that ranged from 8 to 20 French with a preferred range of 12 to 18 French. The inside diameter of the sheath would permit instruments ranging from 6 French to 18 French to pass therethrough, with a preferred range of between 10 and 16 French. The proximal end of the catheter, which is not advanced into a ureter, may be generally larger in diameter to encompass the structure necessary for pushability, torqueability control, and the ability to pass large diameter instruments therethrough. The transluminal access sheath comprises elements that improve on current devices. These improvements include walls that deform plastically, rather than elastomerically. These improvements also include reinforcing structures within the sheath wall, said reinforcing structures having improved radiopaque characteristics. The improvements also include dilator tip shapes that improve guidewire trackability and minimize the potential for damage to adjacent anatomic structures.

One embodiment of the device involves sheath wall construction that is comprised of an inner liner or layer, a middle reinforcing layer, and an outer layer or sleeve. In an embodiment, the inner surface of the inner liner comprises longitudinally oriented valleys and peaks. This construction, called fluting, is intended to minimize contact with devices or objects being passed through the sheath and, in so doing, minimizes resistance or friction. The peaks and/or valleys may be rectangular, rounded, or distinctly “V”-shaped. The fluted construction further permits passage of devices such as ureteroscopes, with less risk of damage due to abrasive particulates becoming wedged between the sheath inner wall and the instrumentation or device being passed therethrough. The aforementioned particulates can potentially cause damage to fragile structures such as lenses and articulating mechanisms by rubbing or being dammed against the front of said fragile structures. The abrasive particulates can cause damage by direct contact with the devices. The fluted inner diameter further provides enhanced irrigation flow even if obstructing devices are in the lumen. These flutes can also serve to increase column strength, and promote fluid transport and drainage through the sheath. The fluted inner liner is fabricated using tubing members that are extruded with the fluted cross-section being created by the extrusion die. The fluted inner liner may also be fabricated using concentric, round extrusions that are heated and re-formed during secondary operations. Such heating and re-forming secondary operations, as well as shape extrusion can be used to create flutes on tubes that are not composite but rather are comprised of a single extrusion. In an embodiment, a special mandrel is used to fabricate the sheath, wherein the outer surface of the mandrel is a fluted mirror image of the flutes created in the inner liner. Thus, when the inner liner is mounted over the mandrel and correctly aligned, the flutes on the sheath will not be melted away when the outer layer is heated and compressed over the composite structure.

In another embodiment, a reinforcing layer is disposed intermediate to the inner and outer layers. The purpose of the reinforcing layer includes crush resistance as well as kink resistance. The reinforcing layer may also be configured to provide torqueability as well as pushability. The reinforcing layer is preferably embedded within the inner and outer layers such that a smooth surface exists on the inner surface of the inner layer and, optionally, on the outer surface of the outer layer. It is beneficial to keep the distance between the adjacent coils of a reinforcing structure substantially near the width of the material used to fabricate the coil. Such close spacing minimizes the amount of roughness on both the inner wall and the outer wall of the sheath. The coil configuration is preferably such that flexibility is not compromised by spacing the adjacent coils too closely. The wire used to form the coil is preferably a flat wire and more preferably a flat wire with rounded, non-sharp edges, borders, or corners. The most preferable coil would be fabricated from an oval wire with no distinct edges at all. The wire is advantageously coated with gold, platinum, platinum iridium, tantalum, or the like to improve the radiopaque density of the coil when visualized under fluoroscopy. The wire is, in an embodiment, fabricated from spring hardness metals such as 304, 316L or other stainless steel, Elgiloy, MP35-N, nitinol, and the like. The wires may also be formed of high strength polymers such as polyamide, polyester, and the like. Such polymer wires are especially in need of the radiopaque coating to enhance their nearly invisible radio-density. The radiopacity of the polymer wires may be enhanced through the use of bismuth compounds or a barium salt, such as barium sulfate, or other radio-dense materials being compounded into the polymer prior to extrusion. Concentrations of barium salts of between 10% and 50% are suitable for radio-density enhancement; however, strength can be lost in this process. Coating the wires with metallic materials using sputter coating, vapor deposition, or dip coating may be the preferred radiopacity enhancing modality. Metallic materials suitable for coating the wires include, but are not limited to, gold, platinum, iridium, tantalum, and the like. The wires are preferably wound over a mandrel, after placing the inner liner over said mandrel, such that the wires are not spring loaded or biased to squeeze inward as this may result in erosion or eruption through the inner layer. Once the coil is wound onto the outside of the inner layer, the coil is secured at its ends and is ready for covering by the outer layer. The securing mechanism, such as tape or a clamp, is, preferably but not always, removed prior to covering by the outer layer.

In another embodiment, the reinforcing layer comprises a polymeric braid, designed to provide degrees of support and shape retention, allowing the sheath cross-section to readjust to body geometry or to the shape of objects pulled through the lumen. This is unlike devices with metallic reinforcement or braid, which will generally have more resistance to bending leading to a greater tendency to be round. Compliance, or the ability of the sheath wall to adjust its cross-sectional characteristics, may allow the removal or passage of large or irregularly shaped stones, instruments, or other materials. Such irregularly shaped materials may have a single dimension greater than the diameter of the sheath and still pass, as long as the orthogonal dimension and overall circumference is less than that of the inner lumen of the sheath. The braided structure has greater torqueability and pushability than a coiled structure, although it may be a less flexible, or bendable, structure. These characteristics make it especially suited to the proximal end of the sheath tubing in a multiple, staged tubing configuration.

All polymeric construction may have potential benefits when used in strong magnetic fields, since they will not inductively heat like metallic reinforcement will. This may be of benefit for devices other than ureteral sheaths; for example, for devices used with processes such as magnetic resonance imaging (MRI) equipment or for collagen shrinking, both of which processes induce significant magnetic fields. Such MRI fields may cause localized heating, which could burn tissue, and create strong dislodgement forces on certain metallic structures. The materials suited for the polymeric braid construction include polyethylene terephthalate (PET), polyamide (Nylon, Kevlar, and the like), and polyethylene naphthalate, (PEN).

In another embodiment, the sheath incorporates drainage holes which fenestrate the sidewalls of the sheath to allow for removal of fluid in the bladder, the drainage of which would otherwise be obstructed, to at least some degree, by the sheath. All polymeric construction (including braided monofilament reinforcement) is advantageous in this configuration since the lack of metal makes it relatively easy to bore drainage holes through the wall without the difficulties or hazards associated with metallic wires. These hazards include sharp wire ends protruding out of the holes such that they might cut tissue or the walls of body lumens, cavities, or vessels through which the catheter or sheath is being passed. In addition to ureteral sheaths, this feature may be of use in specialized drainage devices, like urological stents. The side holes may be advantageously located along the catheter such that they are located within the urinary bladder when the sheath is positioned within the ureter and its distal end located at the region of the renal pelvis. The side holes in the bladder region may provide for improved drainage of fluids during a urological procedure. Of course, the side holes may also be located in the region of the ureter or urethra should that prove beneficial. Side holes for drainage may also be advantageous, for example, in biliary applications. These holes can be drilled completely through the wall of the sheath from the exterior to the inner lumen. The holes can be located at any axial location on the sheath to provide for drainage or fluid flow as desired. The holes can range in diameter from 0.0005 inches to 0.500 inches in diameter, and preferably between 0.005 and 0.050 inches in diameter depending on, and generally not exceeding the catheter or sheath inner diameter. A large size proximal lumen in the sheath, with minimal flow obstruction, will enhance drainage through holes positioned midway along the sheath tubing.

Another embodiment of the transluminal sheath is a staged design wherein materials of different flexibility, stiffness, pushability, torqueability, radiopacity, wall thickness, or other property, may be affixed end to end to form a linearly composite structure. By fusing together different pieces of tubing of different hardness, thickness, and filler compositions, a sheath with variable stiffness or other property can be easily fabricated. This may offer utility for optimizing the sheath for different anatomy and/or different types of procedures. In an advantageous configuration, the sheath is most stiff and has the greatest wall thickness at or near the proximal end. A central region of intermediate stiffness and wall thickness is affixed at the distal end of the stiff region with the central lumens of the two tubes being operably connected. A third region of yet greater flexibility and reduced stiffness is affixed to the distal end of the intermediate central region. The stiffest region may be configured to traverse the urethra, the intermediate region configured to traverse the bladder where sharp bends in an open volume may be encountered, and the third distal region configured to traverse the ureter, which is small in diameter and somewhat tortuous, in one example of this embodiment. The different regions of stiffness can also be created by altering the pitch of the coil reinforcement or the pitch of the braid at various stages along the sheath.

Another embodiment of the invention involves the use of an atraumatic tip at the distal end of the sheath. The atraumatic tip reduces the potential for damage to the ureteral lining during insertion. The atraumatic tip is fabricated from a softer material than the rest of the catheter or is fabricated with thinner walls so that it feels softer to the touch. The atraumatic tip may be affixed to the distal end of the sheath, the distal end of the dilator, or both. The atraumatic tip is tapered inwardly moving in the distal direction. The atraumatic tip may be fabricated from low durometer polymers such as, but not limited to, thermoplastic elastomer, silicone elastomer, polyurethane, latex rubber, C-Flex, and the like. The atraumatic tip may be heat welded, insert molded, adhered, or mechanically affixed to the distal end of the sheath or dilator. The atraumatic tip is configured to taper any step down in dilator or sheath diameter moving distally, so that a shoehorn or gentle taper always meets and coerces the tissue of the body lumen or vessel outward.

In another embodiment, the catheter comprises reinforcing material with a plated or coated layer to enhance radiopacity. This coating is not as advantageous when the reinforcing material is very thick and radiodense. However, when a thin metallic reinforcement is used, allowing for reduced wall thickness of the sheath, the need for radiopacity enhancement becomes advantageous. In a preferred embodiment, a layer of elemental gold, approximately 50 to 500 microns thick is applied to the exterior of the structure making up the reinforcing layer. The gold is applied by dip coating, sputter coating, or other plating process. Materials, other than gold, include barium compounds, platinum, platinum iridium, tantalum, and the like.

An advantageous characteristic of a transluminal sheath is the ability of the sheath to exhibit lubricity. The sheath should pass through the body lumen or vessel with minimal friction. Minimizing friction is accomplished by coating the exterior of the sheath with a lubricious coating such as a hydrophilic hydrogel, silicone oil, or the like. Friction reduction can also occur by fabricating the sheath tissue contact surfaces with materials such as polytetrafluoroethylene (PTFE), FEP, polyethylene, polypropylene, or the like. Further friction reduction can be accomplished by reducing the cross-sectional contact area of the sheath with the tissue. Such surface area reduction can be accomplished by fabricating longitudinally oriented ridges and valleys on the sheath. Such a pattern of ridges and valleys may also be termed flutes. Flutes on the outside diameter of the sheath allow for reduced tissue contact area, and thus a reduced level of friction between the sheath and the tissue. Furthermore, the flutes permit the presence of moisture to access the ureteral lining with the sheath in place, reducing tissue or lining abrasion and irritation. Without the flutes, a squeegee effect can take place, reducing the moisture layer between the sheath and the body lumen or vessel wall and thus increasing friction. A typical flute system can have between 1 and 50 ridges circumferentially distributed around a sheath with an outer diameter of between 8 and 20 French, with a preferred number of between 4 and 20 ridges with corresponding valleys. The ridge to valley height or projection can be in the range of 0.001 inches to 0.030 inches, with a preferred height of 0.002 to 0.010 inches. The height of the flutes is optimized to minimize wall thickness and friction, two generally contradictory requirements.

In another embodiment, the OD of the sheath is rendered slightly rough or dimpled. This wavy surface characteristic is generated using a braided reinforcement surrounded by a polymeric inner and outer layer. The polymer dimples inward between the fibers of the braid to create a type of surface roughness or waviness. This surface waviness reduces the surface area at a given diameter and, thus provides less intimal contact with ureteral lining, allowing moisture to access the lining, thus reducing lining abrasion and irritation. The spacing of the fibers of the braid may be controlled to create the exact surface waviness characteristic desired.

Another embodiment of the sheath comprises radiopaque markers affixed at or near the distal end of the sheath or to other more proximally located regions of the sheath. These radiopaque markers comprise polymer materials doped with radiopaque filler materials such as barium salt, bismuth compounds, tantalum powder, or the like. The polymer materials are heat welded, integral to, or adhered to the distal end of the sheath tubing. In another embodiment, the sheath hub, generally a funnel-shaped structure, may comprise flutes on its outwardly tapering inner surface as well as the inner surface of the hub that runs generally along the long axis of the sheath.

For purposes of summarizing the invention, certain aspects, advantages and novel features of the invention are described herein. It is to be understood that not necessarily all such advantages may be achieved in accordance with any particular embodiment of the invention. Thus, for example, those skilled in the art will recognize that the invention may be embodied or carried out in a manner that achieves one advantage or group of advantages as taught herein without necessarily achieving other advantages as may be taught or suggested herein. These and other objects and advantages of the present invention will be more apparent from the following description taken in conjunction with the accompanying drawings.

### BRIEF DESCRIPTION OF THE DRAWINGS

A general architecture that implements the various features of the invention will now be described with reference to the drawings. The drawings and the associated descriptions are provided to illustrate embodiments of the invention and not to limit the scope of the invention. Throughout the drawings, reference numbers are re-used to indicate correspondence between referenced elements.

FIG. 1 is a front view schematic representation of a urethra, bladder and ureter;

FIG. 2 is a front view schematic representation of the urethra, bladder and ureter with an exemplary access sheath passed into the ureter by way of the urethra;

FIG. 3A illustrates a side view of a transluminal access sheath, according to an embodiment of the invention, with a portion of the sheath shown in cross-section;

FIG. 3B illustrates a side view of an obturator for the transluminal access sheath of FIG. 3A, according to an embodiment of the invention, with a portion of the obturator shown in cross-section;

FIG. 3C illustrates a side view of the obturator of FIG. 3B inserted into the sheath of FIG. 3A;

FIG. 4 is a lateral cross-sectional view of the transluminal access sheath taken through line 4-4 of FIG. 3A;

FIG. 5 is a cross-sectional view similar to FIG. 4 of a modified embodiment of a transluminal access sheath;

FIG. 6 is a side view similar to FIG. 3A of a modified embodiment of a transluminal access sheath;

FIG. 7 is a side view of another embodiment of a transluminal access sheath;

FIG. 8 is a side view of another embodiment of a catheter or sheath inserted into the urinary tract;

FIG. 9 is a side view of an embodiment of an catheter or sheath with a modified embodiment of an obturator inserted therein;

FIG. 10 is a lateral cross-sectional view of an embodiment of catheter or sheath with a material particle disposed therein, the dashed lines showing an un-deformed configuration and the solid lines showing a configuration deformed by the particle therein; and

FIG. 11 illustrates a lateral cross-section of an embodiment of an access sheath comprising an inner layer, a reinforcing layer, an intermediate layer, and an outer layer, according to an embodiment of the invention.

### DETAILED DESCRIPTION OF THE PREFERRED EMBODIMENTS

In the description herein, reference will be made to a catheter or a sheath, can generally be described as being an axially elongate hollow tubular, but not necessarily round, structure having a proximal end and a distal end. The axially elongate structure further has a longitudinal axis and has an internal through lumen that extends from the proximal end to the distal end for the passage of instruments, fluids, tissue, implants, or other materials. As is commonly used in the art of medical devices, the proximal end of the device is that end that is closest to the user, typically a surgeon or interventionalist. The distal end of the device is that end is closest to the patient or is first inserted into the patient. A direction being described as being proximal to a certain landmark will be closer to the surgeon, along the longitudinal axis, and further from the patient than the specified landmark.

The diameter of a catheter or sheath is often measured in “French size” and thus the description herein will also refer to French size. The French size is designed to correspond to the circumference of the catheter in mm and is often useful for catheters that have non-circular cross-sectional configurations. The original measurement of “French” used pi (3.14159 . . . ) as the conversion factor between diameters in mm and French, the system has evolved today to where often the conversion factor is exactly 3.0. For example, a 15 French catheter is 5 mm in diameter.

As will be described in detail below with reference to the figures, one embodiment of the invention comprises an access sheath configured provide access to the ureter, kidney, or bladder. In such an embodiment, the sheath advantageously has an introduction outside diameter that is within the range from about 8 to about 20 French with a preferred range of about 12 to about 18 French. The inside diameter of the sheath would permit instruments ranging from about 6 French to about 18 French to pass therethrough, with a preferred range of between about 10 and about 16 French. The proximal end of the catheter, which is not advanced into a ureter, may be generally larger in diameter to encompass the structure necessary for pushability, torqueability control, and the ability to pass large diameter instruments therethrough. The transluminal access sheath can include elements that improve on current devices. For example, these improvements include walls that deform plastically, rather than elastomerically. These improvements also include reinforcing structures within the sheath wall, said reinforcing structures having improved radiopaque characteristics. The improvements also include dilator tip shapes that improve guidewire trackability and minimize the potential for damage to adjacent anatomic structures.

In one embodiment, the device comprises a sheath wall that is formed from an inner liner or layer, a middle reinforcing layer, and an outer layer or sleeve. In an embodiment, the inner surface of the inner liner comprises longitudinally oriented valleys and peaks. This construction, called fluting, is configured to minimize contact with devices or objects being passed through the sheath and, in so doing, minimizes resistance or friction. The peaks and/or valleys may be rectangular, rounded, or distinctly “V”-shaped. The fluted construction further permits passage of devices such as ureteroscopes, with less risk of damage due to abrasive particulates becoming wedged between the sheath inner wall and the instrumentation or device being passed therethrough. The aforementioned particulates can potentially cause damage to fragile structures such as lenses and articulating mechanisms by rubbing or being dammed against the front of said fragile structures. The abrasive particulates can cause damage by direct contact with the devices. The fluted inner diameter further provides enhanced irrigation flow even if obstructing devices are in the lumen. These flutes can also serve to increase column strength, and promote fluid transport and drainage through the sheath. The fluted inner liner is fabricated using tubing members that are extruded with the fluted cross-section being created by the extrusion die. The fluted inner liner may also be fabricated using concentric, round extrusions that are heated and re-formed during secondary operations. Such heating and re-forming secondary operations, as well as shape extrusion can be used to create flutes on tubes that are not composite but rather are comprised of a single extrusion. In an embodiment, a special mandrel is used to fabricate the sheath, wherein the outer surface of the mandrel is a fluted mirror image of the flutes created in the inner liner. Thus, when the inner liner is mounted over the mandrel and correctly aligned, the flutes on the sheath will not be melted away when the outer layer is heated and compressed over the composite structure.

In another embodiment, a reinforcing layer is disposed intermediate to the inner and outer layers. The purpose of the reinforcing layer includes crush resistance as well as kink resistance. The reinforcing layer may also be configured to provide torqueability as well as pushability. The reinforcing layer is preferably embedded within the inner and outer layers such that a smooth surface exists on the inner surface of the inner layer and, optionally, on the outer surface of the outer layer. It is beneficial to keep the distance between the adjacent coils of a reinforcing structure substantially near the width of the material used to fabricate the coil. Such close spacing minimizes the amount of roughness on both the inner wall and the outer wall of the sheath. The coil configuration is preferably such that flexibility is not compromised by spacing the adjacent coils too closely. The wire used to form the coil is preferably a flat wire and more preferably a flat wire with rounded, non-sharp edges, borders, or corners. The most preferable coil would be fabricated from an oval wire with no distinct edges at all. The wire is advantageously coated with gold, platinum, platinum iridium, tantalum, or the like to improve the radiopaque density of the coil when visualized under fluoroscopy. The wire is, in an embodiment, fabricated from spring hardness metals such as 304, 316L or other stainless steel, Elgiloy, MP35-N, nitinol, and the like. The wires may also be formed of high strength polymers such as polyamide, polyester, and the like. Such polymer wires are especially in need of the radiopaque coating to enhance their nearly invisible radio-density. The radiopacity of the polymer wires may be enhanced through the use of bismuth compounds or a barium salt, such as barium sulfate, or other radio-dense materials being compounded into the polymer prior to extrusion. Concentrations of barium salts of between 10% and 50% are suitable for radio-density enhancement; however, strength can be lost in this process. Coating the wires with metallic materials using sputter coating, vapor deposition, or dip coating may be the preferred radiopacity enhancing modality. Metallic materials suitable for coating the wires include, but are not limited to, gold, platinum, iridium, tantalum, and the like. The wires are preferably wound over a mandrel, after placing the inner liner over said mandrel, such that the wires are not spring loaded or biased to squeeze inward as this may result in erosion or eruption through the inner layer. Once the coil is wound onto the outside of the inner layer, the coil is secured at its ends and is ready for covering by the outer layer. The securing mechanism, such as tape or a clamp, is, preferably but not always, removed prior to covering by the outer layer.

In another embodiment, the reinforcing layer comprises a polymeric braid, designed to provide degrees of support and shape retention, allowing the sheath cross-section to readjust to body geometry or to the shape of objects pulled through the lumen. This is unlike devices with metallic reinforcement or braid, which will generally have more resistance to bending leading to a greater tendency to be round. Compliance, or the ability of the sheath wall to adjust its cross-sectional characteristics, may allow the removal or passage of large or irregularly shaped stones, instruments, or other materials. Such irregularly shaped materials may have a single dimension greater than the diameter of the sheath and still pass, as long as the orthogonal dimension and overall circumference is less than that of the inner lumen of the sheath. The braided structure has greater torqueability and pushability than a coiled structure, although it may be a less flexible, or bendable, structure. These characteristics make it especially suited to the proximal end of the sheath tubing in a multiple, staged tubing configuration.

All polymeric construction may have potential benefits when used in strong magnetic fields, since they will not inductively heat like metallic reinforcement will. This may be of benefit for devices other than ureteral sheaths; for example, for devices used with processes such as magnetic resonance imaging (MRI) equipment or for collagen shrinking, both of which processes induce significant magnetic fields. Such MRI fields may cause localized heating, which could burn tissue, and create strong dislodgement forces on certain metallic structures. The materials suited for the polymeric braid construction include polyethylene terephthalate (PET), polyamide (Nylon, Kevlar, and the like), and polyethylene naphthalate, (PEN).

In another embodiment, the sheath incorporates drainage holes which fenestrate the sidewalls of the sheath to allow for removal of fluid in the bladder, the drainage of which would otherwise be obstructed, to at least some degree, by the sheath. All polymeric construction (including braided monofilament reinforcement) is advantageous in this configuration since the lack of metal makes it relatively easy to bore drainage holes through the wall without the difficulties or hazards associated with metallic wires. These hazards include sharp wire ends protruding out of the holes such that they might cut tissue or the walls of body lumens, cavities, or vessels through which the catheter or sheath is being passed. In addition to ureteral sheaths, this feature may be of use in specialized drainage devices, like urological stents. The side holes may be advantageously located along the catheter such that they are located within the urinary bladder when the sheath is positioned within the ureter and its distal end located at the region of the renal pelvis. The side holes in the bladder region may provide for improved drainage of fluids during a urological procedure. Of course, the side holes may also be located in the region of the ureter or urethra should that prove beneficial. Side holes for drainage may also be advantageous, for example, in biliary applications. These holes can be drilled completely through the wall of the sheath from the exterior to the inner lumen. The holes can be located at any axial location on the sheath to provide for drainage or fluid flow as desired. The holes can range in diameter from 0.0005 inches to 0.500 inches in diameter, and preferably between 0.005 and 0.050 inches in diameter depending on, and generally not exceeding the catheter or sheath inner diameter. A large size proximal lumen in the sheath, with minimal flow obstruction, will enhance drainage through holes positioned midway along the sheath tubing.

Another embodiment of the transluminal sheath is a staged design wherein materials of different flexibility, stiffness, pushability, torqueability, radiopacity, wall thickness, or other property, may be affixed end to end to form a linearly composite structure. By fusing together different pieces of tubing of different hardness, thickness, and filler compositions, a sheath with variable stiffness or other property can be easily fabricated. This may offer utility for optimizing the sheath for different anatomy and/or different types of procedures. In an advantageous configuration, the sheath is most stiff and has the greatest wall thickness at or near the proximal end. A central region of intermediate stiffness and wall thickness is affixed at the distal end of the stiff region with the central lumens of the two tubes being operably connected. A third region of yet greater flexibility and reduced stiffness is affixed to the distal end of the intermediate central region. The stiffest region may be configured to traverse the urethra, the intermediate region configured to traverse the bladder where sharp bends in an open volume may be encountered, and the third distal region configured to traverse the ureter, which is small in diameter and somewhat tortuous, in one example of this embodiment. The different regions of stiffness can also be created by altering the pitch of the coil reinforcement or the pitch of the braid at various stages along the sheath.

Another embodiment of the invention involves the use of an atraumatic tip at the distal end of the sheath. The atraumatic tip reduces the potential for damage to the ureteral lining during insertion. The atraumatic tip is fabricated from a softer material than the rest of the catheter or is fabricated with thinner walls so that it feels softer to the touch. The atraumatic tip may be affixed to the distal end of the sheath, the distal end of the dilator, or both. The atraumatic tip is tapered inwardly moving in the distal direction. The atraumatic tip may be fabricated from low durometer polymers such as, but not limited to, thermoplastic elastomer, silicone elastomer, polyurethane, latex rubber, C-Flex, and the like. The atraumatic tip may be heat welded, insert molded, adhered, or mechanically affixed to the distal end of the sheath or dilator. The atraumatic tip is configured to taper any step down in dilator or sheath diameter moving distally, so that a shoehorn or gentle taper always meets and coerces the tissue of the body lumen or vessel outward.

In another embodiment, the catheter comprises reinforcing material with a plated or coated layer to enhance radiopacity. This coating is not as advantageous when the reinforcing material is very thick and radiodense. However, when a thin metallic reinforcement is used, allowing for reduced wall thickness of the sheath, the need for radiopacity enhancement becomes advantageous. In a preferred embodiment, a layer of elemental gold, approximately 50 to 500 microns thick is applied to the exterior of the structure making up the reinforcing layer. The gold is applied by dip coating, sputter coating, or other plating process. Materials, other than gold, include barium compounds, platinum, platinum iridium, tantalum, and the like.

An advantageous characteristic of a transluminal sheath is the ability of the sheath to exhibit lubricity. The sheath should pass through the body lumen or vessel with minimal friction. Minimizing friction is accomplished by coating the exterior of the sheath with a lubricious coating such as a hydrophilic hydrogel, silicone oil, or the like. Friction reduction can also occur by fabricating the sheath tissue contact surfaces with materials such as polytetrafluoroethylene (PTFE), FEP, polyethylene, polypropylene, or the like. Further friction reduction can be accomplished by reducing the cross-sectional contact area of the sheath with the tissue. Such surface area reduction can be accomplished by fabricating longitudinally oriented ridges and valleys on the sheath. Such a pattern of ridges and valleys may also be termed flutes. Flutes on the outside diameter of the sheath allow for reduced tissue contact area, and thus a reduced level of friction between the sheath and the tissue. Furthermore, the flutes permit the presence of moisture to access the ureteral lining with the sheath in place, reducing tissue or lining abrasion and irritation. Without the flutes, a squeegee effect can take place, reducing the moisture layer between the sheath and the body lumen or vessel wall and thus increasing friction. A typical flute system can have between 1 and 50 ridges circumferentially distributed around a sheath with an outer diameter of between 8 and 20 French, with a preferred number of between 4 and 20 ridges with corresponding valleys. The ridge to valley height or projection can be in the range of 0.001 inches to 0.030 inches, with a preferred height of 0.002 to 0.010 inches. The height of the flutes is optimized to minimize wall thickness and friction, two generally contradictory requirements.

In another embodiment, the OD of the sheath is rendered slightly rough or dimpled. This wavy surface characteristic is generated using a braided reinforcement surrounded by a polymeric inner and outer layer. The polymer dimples inward between the fibers of the braid to create a type of surface roughness or waviness. This surface waviness reduces the surface area at a given diameter and, thus provides less intimal contact with ureteral lining, allowing moisture to access the lining, thus reducing lining abrasion and irritation. The spacing of the fibers of the braid may be controlled to create the exact surface waviness characteristic desired.

Another embodiment of the sheath comprises radiopaque markers affixed at or near the distal end of the sheath or to other more proximally located regions of the sheath. These radiopaque markers comprise polymer materials doped with radiopaque filler materials such as barium salt, bismuth compounds, tantalum powder, or the like. The polymer materials are heat welded, integral to, or adhered to the distal end of the sheath tubing. In another embodiment, the sheath hub, generally a funnel-shaped structure, may comprise flutes on its outwardly tapering inner surface as well as the inner surface of the hub that runs generally along the long axis of the sheath.

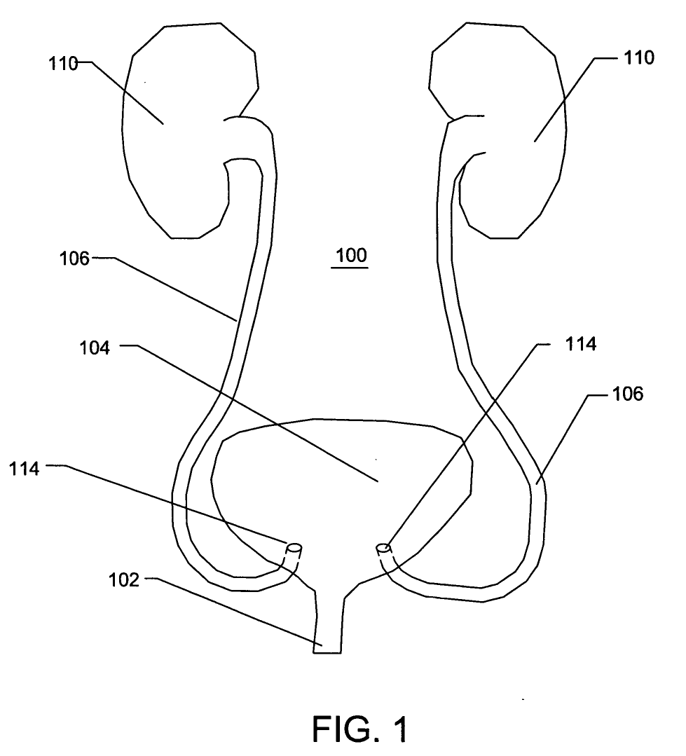

FIG. 1 is a schematic frontal illustration of a urinary system 100 of the human comprising a urethra 102, a bladder 104, a plurality of ureters 106, a plurality of kidneys 110 and a plurality of entrances 114 to the ureter 106 from the bladder 106. In this illustration, the left anatomical side of the body is toward the right of the illustration.

Referring to FIG. 1, the urethra 102 is lined on its interior by urothelium. Generally, the internal surfaces of the urethra 102, the bladder 104, and ureters 106 are considered mucosal tissue. The urethra 102 is relatively short in women and may be long in men since it runs through the entire length of the penis. The circumference of the unstretched urethra 102 is generally in the range of π (pi) times the urethral diameter (e.g. 8 mm), or 24 mm, although the urethra 102 generally approximates the cross-sectional shape of a slit when no fluid or instrumentation is resident therein. The bladder 104 has the capability of holding between 100 and 300 cc of urine or more. The volume of the bladder 104 increases and decreases with the amount of urine that fills therein. During a urological procedure, saline is often infused into the urethra 102 and bladder 104 thus filling the bladder 104. The general shape of the bladder 104 is that of a funnel with a dome shaped top. Nervous sensors detect muscle stretching around the bladder 104 and a person generally empties their bladder 104, when it feels full, by voluntarily relaxing the sphincter muscles that surround the urethra 102.

The ureters 106 operably connect the kidneys 110 to the bladder 104 and permit drainage of urine that is removed from the blood by the kidneys 110 into the bladder 104. The diameter of the ureters 106 in their unstretched configuration approximates a round tube with a 4 mm diameter, although their unstressed configuration may range from round to slit-shaped. The ureters 106 and the urethra 102 are capable of some expansion with the application of internal forces such as a dilator, etc. The entrance 114 to each of the normally two ureters 106 is located on the wall of the bladder 104 in the lower region of the bladder 104.

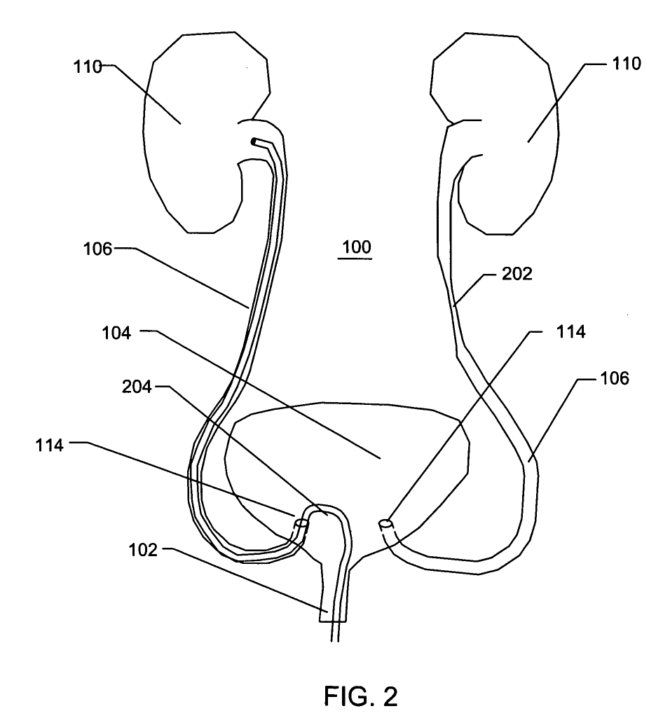

FIG. 2 is a schematic frontal illustration, looking in the posterior direction from the anterior direction, of the urinary system 100 comprising the urethra 102, the bladder 104, a plurality of ureters 106 having entrances 114, a plurality of kidneys 110, a stricture 202 in the left ureter, and further comprising a catheter 204 extending from the urethra 102 into the right kidney 110. In this illustration, the left anatomical side of the body is toward the right of the illustration.

Referring to FIG. 2, the stricture 202 may be the result of a pathological condition such as an infection. The stricture may also be the result of iatrogenic injury such as that attributed to a surgical instrument or catheter that caused damage to the wall of the ureter 106. The stricture 202 may be surrounded by fibrous tissue and may prevent the passage of instrumentation that would normally have passed through a ureter 106. The stricture 202 is a narrowing of the body lumen or vessel and may have a diameter of 10 French of less. The catheter 204 is exemplary of the type used to access the ureter 106 and the kidney 110, having been passed transurethrally into the bladder 104 and on into the ureter 106. A catheter routed from the urethra 102 into one of the ureters 106 may turn a sharp radius within the open unsupported volume of the bladder 104. The radius of curvature necessary for a catheter to turn from the urethra 102 into the ureter 106 may be between 1 cm and 10 cm and in most cases between 1.5 cm and 5 cm. The catheter is generally first routed into the ureter 106 along a guidewire that is placed using a rigid cystoscope. The rigid cystoscope, once it is introduced, straightens out the urethra 102 and is aimed close to the entrance 114 to the ureter 106 to facilitate guidewire placement through the working lumen of the cystoscope.

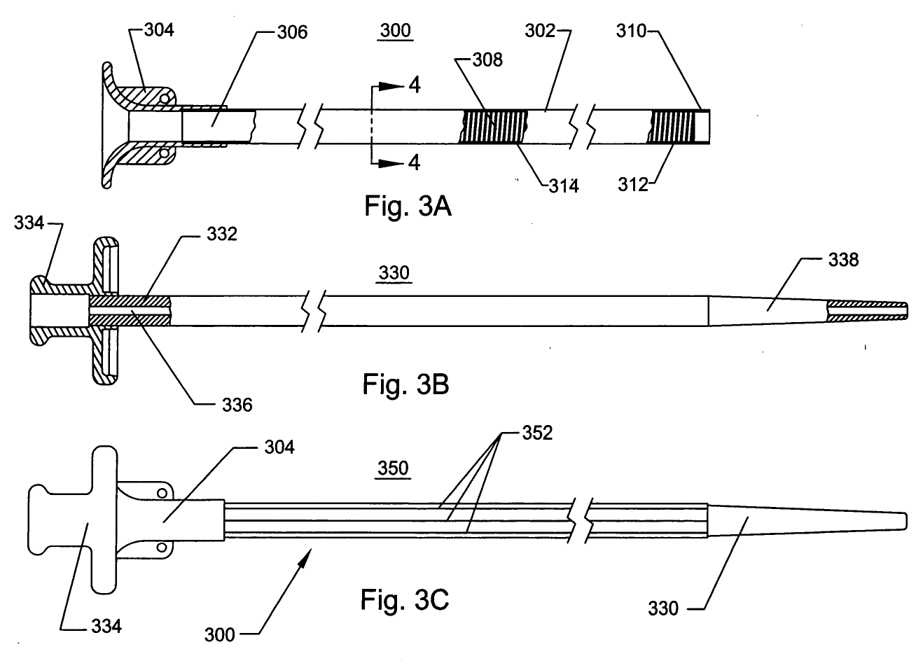

FIG. 3A illustrates a longitudinal, or side, partial cross-sectional view of an embodiment of a transluminal access sheath 300 adapted for use in the urinary system 100 of FIGS. 1 and 2. The sheath 300 comprises a sheath tube 302, a sheath hub 304, a central through lumen 306, and a distal tip 310. As will be explained in more detail below, in the illustrated embodiment, the sheath tube 302 preferably comprises a reinforcing layer 308, an inner layer 314, and an outer layer 312.

Referring to FIG. 3A, the sheath tube 304 can be a composite structure comprising the inner layer 314 fused to the outer layer 312, with the reinforcing layer 308 sandwiched therebetween. Preferably, no part of the reinforcing layer 308 projects beyond the boundary of the inner layer 314 or the outer layer 312. The sheath hub 304 is affixed or coupled to the proximal end of the sheath tube 302 and the distal tip 310 is affixed or coupled to the distal end of the sheath tube 302. The inner layer 314, the outer layer 312, or both, may be pre-treated using plasma discharge or other process to enhance the mechanical interlocking properties of one layer to another, especially if dissimilar materials are used. For example, if a PTFE inner layer 314 is used, pre-treatment will increase the bond strength between the inner layer 314 and an outer layer 312, which is fabricated from, among other options, polyamide or polyurethane.

The reinforcing structure 308 can be a coil of round or flat wire. If flat wire is used for the reinforcing structure, it is preferable that the edges of the wire be rounded so as not to present sharpness, which could erode the sheath tube 302. The wire can comprise stainless steel such as 304 or 316L, titanium, nitinol, cobalt nickel alloy, or the like. The wire can further comprise polymers such as, but not limited to, PET, polyamide, PEN, or the like. The reinforcing structure 308 can be a coil, as shown in FIG. 3A, or it may be a braid; or it may be a simple series of loops. If the reinforcing structure 308 is formed from a coil, it is desirable that the spacing between the adjacent coil members be approximately the same as the width of the wire to minimize dimpling of the inner layer 314 between coil members.

The inner layer 314 and the outer layer 312 can be fabricated from polymers including, but not limited to, polyethylene, high density polyethylene, low density polyethylene, high density-low density blends of polyethylene, FEP, PTFE, polyurethane, PEBAX, Hytrel, or the like. The inner layer 314, the outer layer 312, or both may be coated with a hydrophilic hydrogel, silicone oil, or other biocompatible friction reducing agent. Coatings may be ionicatly bonded, covalently bonded, or not bonded at all to the surface of the sheath tube 302.

The radiopacity of the reinforcing layer 308, the inner layer 314 and/or the outer layer 312 may be enhanced through the use of bismuth compounds or a barium salt, such as barium sulfate, or other radio-dense materials being compounded into the polymer prior to extrusion. Concentrations of barium or bismuth salts of between 10% and 50% are suitable for radio-density enhancement; however, strength can be lost in this process. Coating the wires, either metal or polymeric wires, with metallic materials using sputter coating, vapor deposition, or dip coating may be the preferred radiopacity enhancing modality. Metallic materials suitable for coating the wires include, but are not limited to, gold, platinum, iridium, tantalum, and the like.

The distal tip 310 may be fabricated from soft, elastomeric materials such as C-Flex, silicone rubber, latex rubber, polyurethane, or the like, or it may be fabricated from polyethylene, polypropylene, PTFE, FEP, or the like. The soft embodiment of the tip 310 can have a hardness range of Shore 5 A to Shore 85 A. Wall thicknesses can further be used to modify the overall flexibility and softness of the tip once the hardness of the material has been selected. Soft tips 310 will appear even softer if they are made thinner. The distal tip 310 may be bonded or it may be welded to the sheath tube 302. The distal tip 310 may further be a simple extension of the sheath tube 302 with the reinforcing layer 308 being omitted. The distal tip 310 can further be an extension of the inner layer 314 or the outer layer 312. The outer surface of the distal tip 310 is tapered inward moving distally to minimize or eliminate any sharp transition zones at the distal end of the distal tip 310. The distal tip 310 may further comprise radiopaque markers embedded therein or compounded therein as specified for the inner layer 314 and outer layer 312.

FIG. 3B illustrates a dilator or obturator 330, suitable for filling or plugging the central lumen or inner diameter of the sheath 300 of FIG. 3A. The dilator or obturator 330 comprises a dilator tube 332, a dilator hub 334, a central lumen 336, and a tapered distal tip 338.

Referring to FIG. 3B, the dilator tube 332 in the illustrated embodiment is generally unreinforced elastomeric tubing and is affixed by bonding or welding to the dilator hub 334. The tapered distal tip 338 is integral to the dilator tube 332, although it could be welded or bonded thereon, if desired. The central lumen 336 is generally integral to the dilator tube 332 and is generally formed at the time when the dilator tube 332 is extruded or fabricated, if a composite structure is used to fabricate the dilator tube 332. The dilator tube 332 can comprise longitudinally oriented or spiral cut ridges and adjacent valleys, termed flutes (not shown). These flutes can be added to the outer diameter of the dilator tube 332 during the extrusion process or through the use of a secondary operation. The flutes serve the function of promoting fluid transport and minimizing friction between the dilator/obturator tube 332, and the internal diameter of the sheath tube 302.

The tapered distal tip 338 may comprise a single taper, or it may comprise a more complex shape including one or more tapers and a plurality of cylindrical non-tapered regions as will be described in more detail below. The distal most part of the tapered distal tip 338 should track easily over a guidewire. Guidewires suitable for specific procedures generally dictate the level of trackability of the distal tip. For example, in ureteral applications, a 0.035 or 0.038 inch diameter guidewire is generally used. The dilator tubing 3302 is generally fabricated from elastomeric materials such as Hytrel, polyurethane, C-Flex, silicone elastomer, and the like. The dilator tubing 3302 may be coated with a hydrophilic hydrogel, silicone oil or the like to enhance lubricity. The dilator tubing 3302 may also be alloyed with radiopaque fillers to enhance visualization under fluoroscopy. The material of the distal tip 338 can be made softer or harder than the material of the dilator tubing 332. In an embodiment, the distal tip 338 is fabricated from a thermoplastic elastomer and is welded to a harder Hytrel dilator tube 332. Extra softness in the dilator tip may enhance trackability over a guidewire and render the distal end of the system less traumatic to tissue.

FIG. 3C illustrates the sheath 300 of FIG. 3A with the dilator 330 of FIG. 3B inserted therein forming a sheath/dilator composite structure 350. The dilator hub 334 is snapped onto the sheath hub 304, thus providing a positive mechanical engagement that can be reversed or eliminated at any desired time by simple mechanical force. The tip of the dilator 330 is shown protruding beyond the distal end of the sheath 300. The engagement of the dilator hub 334 to the sheath hub 304 can be created using a relief in the inside of the dilator hub 334, or it can be created using other latches or connectors. It is preferable that the dilator hub 334 has at least three points of engagement with the sheath hub 304 to prevent side-to-side movement and unintentional disengagement, which could cause the dilator 330 to separate form the sheath 300 during the procedure, an undesirable event because the entire structure 350 should be unitary during insertion to minimize tissue damage. The sheath 300 can optionally comprise a plurality of flutes 352 running longitudinally as shown, or in a spiral or rifled fashion, disposed on the exterior surface as shown, the interior surface, or both. Referring to FIG. 3B, the obturator or dilator 300 can also comprise the flutes 352 on the exterior surface of the dilator tube 332 or the dilator tip 338.

The proximal end of the sheath dilator assembly 350 comprises the sheath hub 304 and the dilator hub 334. In an embodiment, the dilator hub 334 is keyed so that when it is interfaced to, or attached to, the sheath hub 304, the two hubs 304 and 334 cannot rotate relative to each other. This is beneficial so that the dilator 330 does not become twisted due to inadvertent rotation of the dilator hub 334 relative to the sheath hub 304. This, the anti-rotation feature of the two hubs 304 and 334 is advantageous. The anti-rotation features could include mechanisms such as, but not limited to, one or more keyed tab on the dilator hub 334 and one or more corresponding keyed slot in the sheath hub 304. Axial separation motion between the dilator hub 334 and the sheath hub 304 easily disengages the two hubs 304 and 334 while rotational relative motion is prevented by the sidewalls of the tabs and slots. A draft angle, for example 1 to 10 degrees, on the sidewalls of the tabs and the slots further promotes engagement and disengagement of the anti-rotation feature. In another embodiment, the sheath hub 304 is releaseably affixed to the dilator hub 334 so the two hubs 304 and 334 are coaxially aligned and prevented from becoming inadvertantly disengaged or separated laterally. In this embodiment, the two hubs 304 and 334 are connected at a minimum of 3 points, which prevent lateral relative motion in both of two substantially orthogonal axes. In a preferred embodiment, the two hubs 304 and 334 are engaged substantially around their full 360-degree perimeter. Manual pressure is sufficient to snap or connect the two hubs 304 and 334 together as well as to separate the two hubs 304 and 334. In another embodiment, the distal end of the sheath hub 304 is configured to taper into the sheath tubing 306 so that the sheath hub 304 distal end can be advanced into the urethral meatus.

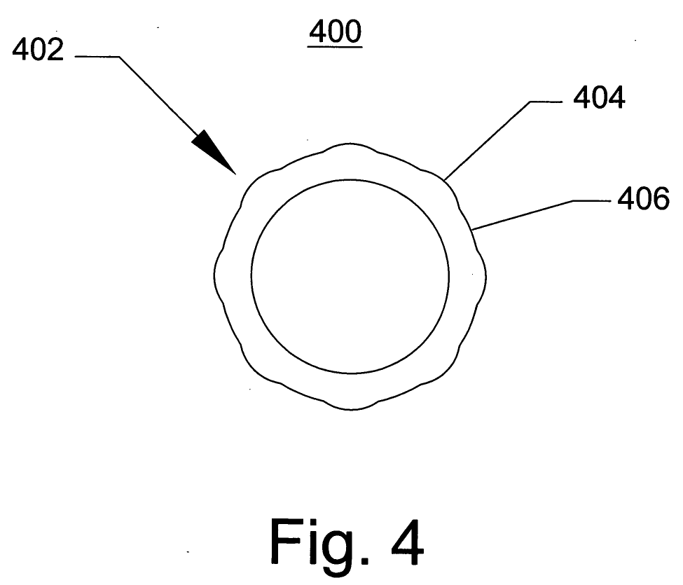

FIG. 4 illustrates a transluminal catheter or sheath tube 400 that is fluted along its external surface. The flutes 402 further comprise longitudinally oriented ridges 404 and valleys 406. The ridges 404 and the valleys 406 are integral to the sheath tube 400 although they could be created by adhering or welding longitudinally oriented runners onto the exterior of the sheath tube 400. The distance between the top of the ridge 404 and the bottom of the valley 406 may vary between 0.0005 inches and 0.020 inches. Preferably, the peak to valley distance varies between 0.001 and 0.010 inches. The transition from ridge 404 to valley 406 may be sharp or it may be rounded, as may the peak of the ridge 404 or the bottom of the valley 406. FIG. 4 illustrates eight flutes 402 on the sheath tube 400 which can have an outer diameter ranging between 0.050 and 0.275 inches. The number of flutes may vary, however, between 1 and 40 for a sheath with an outer diameter of 0.005 inches to a diameter of 0.5 inches.

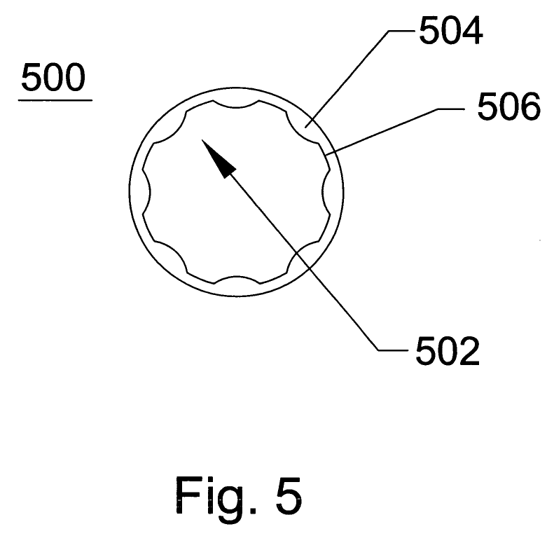

FIG. 5 illustrates a transluminal catheter or sheath tube 500 that is fluted along its internal surface. The flutes 502 further comprise longitudinally oriented ridges 504 and valleys 506. The ridges 504 and the valleys 506 are integral to the sheath tube 500 although they could be created by adhering or welding longitudinally oriented runners onto the interior of the sheath tube 500. The distance between the top of the ridge 504 and the bottom of the valley 506 may vary between 0.0005 inches and 0.020 inches. Preferably, the peak to valley distance varies between 0.001 and 0.010 inches. The transition from ridge 504 to valley 506 may be sharp or it may be rounded, as may the peak of the ridge 504 or the bottom of the valley 506. FIG. 5 illustrates eight flutes 502 on the inner wall of the sheath tube 500. The sheath, in this embodiment can have an internal diameter ranging between 0.070 inches and 0.250 inches. The number of flutes may vary, however, between 1 and 40 for a sheath with an internal diameter of 0.005 inches to a diameter of 0.5 inches.

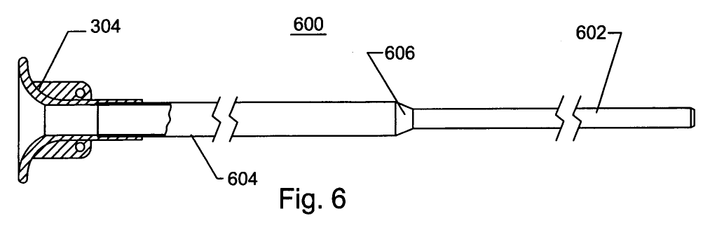

FIG. 6 illustrates a side view of a sheath 600 comprising a proximal section 604, a transition zone 606, and a distal section 602, each of which occupy a different longitudinally located region of the sheath 600. The transition zone 606 between the proximal section 604 and the distal section 602 is tapered to reduce trauma and the chance of catching on tissue when the sheath 600 is inserted into the patient. The proximal section 604 is affixed to the transition zone 606, as is the distal section 602. The proximal end of the proximal section 604 is affixed to the sheath hub 304. In this embodiment, the proximal section 604 has different mechanical properties than the distal section 604. The proximal section 604 is preferably a composite structure with less flexibility than the distal section 602. The proximal section 604 generally maintains higher column strength than the distal section 602 and is generally more torqueable. The different characteristics of the proximal section 604 relative to the distal section 602 are achieved by using polymers with higher hardness and stiffness. Furthermore, the reinforcing layer in the proximal section 604 may be braided and, thus be stiffer than a coil reinforcement, for example, used in the distal section 602. Different characteristics may also be achieved by using a larger wall thickness, for example, in the proximal section 604 than in the distal section 602. Parameters that may differ between the proximal section 604 and the distal section 602 include, but are not limited to, radiopacity (e.g. increasing moving distally), flexibility (e.g. increasing moving distally), torqueability (e.g. relatively constant), column strength (e.g. decreasing moving distally), hoop strength (e.g. decreasing moving distally), permeability or porosity (e.g. increasing moving distally), conductivity, and the like. It may be advantageous to have one or more transition regions 606 with a transition region required for each adjacent pair of mechanically distinct regions. Furthermore, it can be advantageous for the sheath 600 to comprise between 2 and 10 regions of different characteristics or operating parameters, with between two and four regions achieving most of the desirable properties. Variability in flexibility may further be achieved by winding a coil-reinforcing layer with different pitches. For example, the pitch at the proximal end of the sheath may be 0.02 inches per turn while the pitch at the distal end may be larger, for example 0.040 inches per turn.

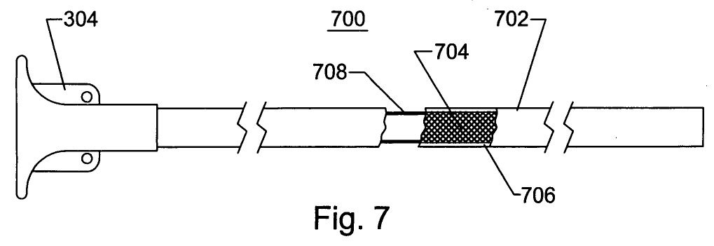

FIG. 7 illustrates a side breakaway view of a transluminal sheath 700 comprising a sheath hub 304, and a sheath tube 702. The sheath tube 702 further comprises a reinforcing mesh 704, an outer sheath layer 706 and an inner sheath layer 708. The reinforcing mesh 704 is, in this embodiment, a braided structure that is sandwiched between the outer sheath layer 706 and the inner sheath layer 708, the latter two of which are fused together through the reinforcing mesh 704. In this embodiment, since the inner sheath layer 708 and the outer sheath layer 706 are welded or fused together through the reinforcing mesh 704, the mesh 704 is not able to expand or contract in either diameter or length, resulting in a structure that is flexible but stable in diameter as well as being pushable and torqueable. The mesh 704 is preferably fabricated from polymeric strands of material such as, but not limited to, PET, PEN, Kevlar, polyamide, polyurethane, polyethylene, polypropylene, and the like. The mesh 704 could also be fabricated from metal such as, but not limited to, nitinol, titanium, stainless steel, Elgiloy, and the like. However, the polymeric braid is advantageous in that it is somewhat more flexible than the metal braid and can deform to an out-of round condition in a resilient or semi-resilient fashion. The mesh 704, whether metal or plastic, can be coated with a radiopaque material such as, but not limited to, tantalum, gold, silver, platinum, iridium, and the like at thicknesses of between 50 and 500 microns to enhance visibility under fluoroscopy.

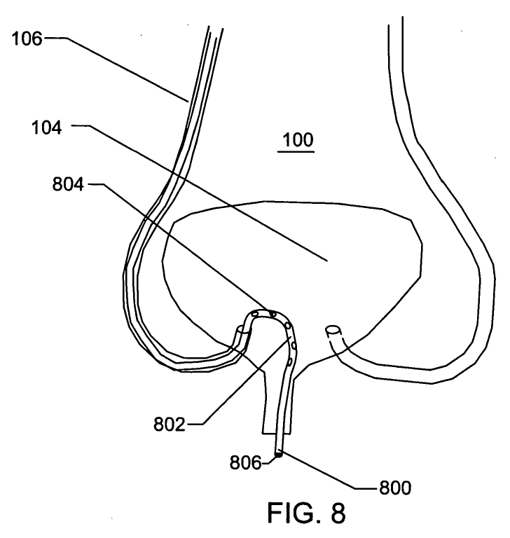

FIG. 8 illustrates a side view of a catheter or sheath 800 inserted into a urinary tract, wherein the catheter or sheath tubing 802 comprises holes or fenestrations 804. The holes or fenestrations 804 operably communicate between the central lumen 806 and the external environment of the sheath 800. Thus, it is possible to withdraw fluids into the central lumen 806 through holes or fenestrations 804 and have those fluids be withdrawn from the proximal end of the sheath 800. It is further possible to inject fluids at the proximal end of the sheath 800 and have those fluids exit either at the distal tip of the sheath 800 or through the holes or fenestrations 804. It is beneficial to plug or occlude the distal tip of the sheath 800 to force fluid exit at the holes or fenestrations 804.

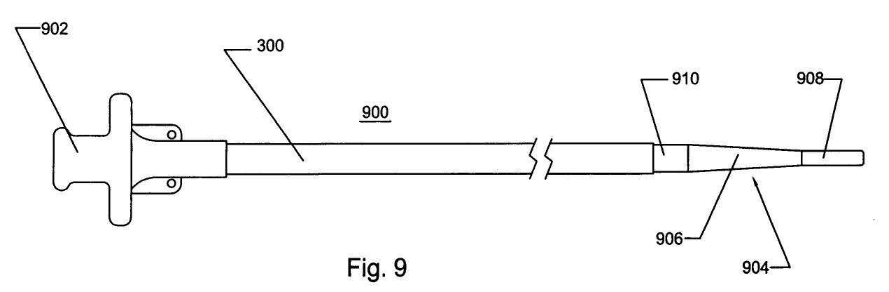

FIG. 9 illustrates a side view of a catheter or sheath 900 with an obturator or dilator 902 comprising a tissue dilating tip 904, wherein the tip 904 comprises a complex shape rather than a simple taper. In this embodiment, the tissue-dilating tip 904 comprises a distal flat region 908, an intermediate taper region 906, and a proximal flat region 910. The tissue-dilating tip 904 is a generally integral structure wherein the distal flat region 908, the intermediate taper 906 and the proximal flat region 910 are heat formed or molded from a single piece of material, typically the same material used to make up the dilator or obturator shaft (not shown). The materials used to make the tissue dilating tip 904 include, but are not limited to, polyethylene, polypropylene, polyamide, polyurethane, Hytrel, Pebax, silicone elastomer, C-Flex, and the like. The distal flat region 908 is configured to track over a guidewire and bend easily around corners with the guidewire. The intermediate taper 904 is configured as a strain relief becoming increasingly stiffer moving proximally so that the large diameter sheath 300 is coerced to track the guidewire. The proximal flat region 910 is configured to fit snugly with the inner lumen (not shown) of the sheath 300 so as to minimize gaps and edges as well as to coerce the sheath 300 to follow the guidewire. The intermediate taper 904 can also be configured with multiple tapers, which are fitted to guidewires or sheaths 300 of different stiffness. Furthermore, intermediate flat regions (not shown) can be added within the region of intermediate taper 904 to further tailor the stiffness increase to match the sheath 300 and guidewire.

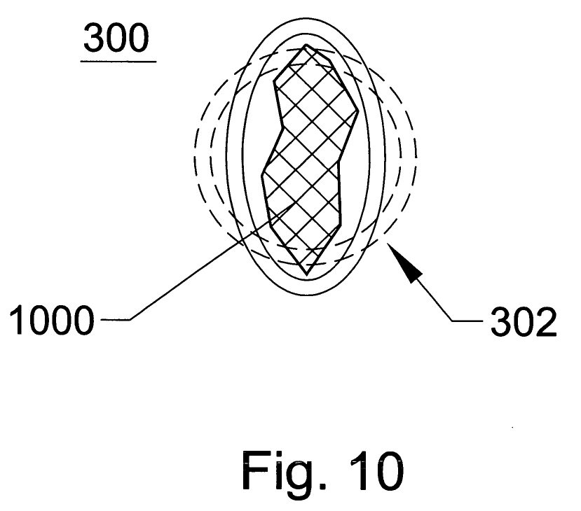

FIG. 10 is a cross-sectional view of the tubing 302 comprising the catheter or sheath 300 with a material particle 1000 disposed therein, wherein the material particle 1000 has deformed the tubing 302 cross-section to accommodate a dimension larger than the diameter of the sheath 300. The overall circumference of the tubing 302 remains constant. The tubing 302 is now distorted and has become larger in one direction and smaller in another direction to accommodate the large object 1000. A ghost of the original undistorted tubing 302 is shown as a dotted line. The central lumen 306 is no longer round but oval or ellipsoidal, in an embodiment. Note that the material particle 1000 would not fit in the original tubing 302 but now fits within the distorted tubing 302. This distortion is made possible by use of flexible walls with high flexibility. While hoop strength may or may not be retained, flexibility is an advantageous characteristic for the tubing 302.

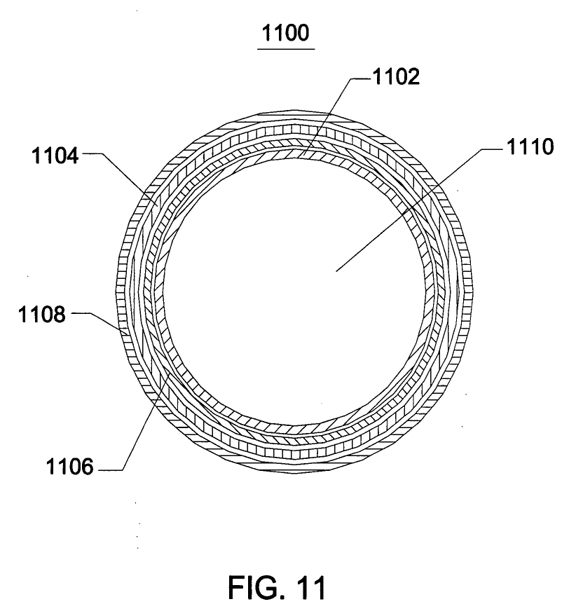

FIG. 11 illustrates a lateral cross-section of a sheath tube 1100 comprising an inner layer 1102, a reinforcing layer 1106, an elastomeric layer 1104, and an outer layer 1108. The sheath tube 1100 comprises a central lumen 1110. The elastomeric layer 1104 can be disposed outside the reinforcing layer 1106, inside the reinforcing layer 1106, or both inside and outside the reinforcing layer 1106. The elastomeric layer 1104 is fabricated from silicone elastomer, thermoplastic elastomer such as C-Flex™, a trademark of Concept Polymers, polyurethane, or the like. The hardness of the elastomeric layer 1104 can range from Shore 10 A to Shore 90 A with a preferred range of Shore 50 A to Shore 70 A. The inner layer 1102 and the outer layer 1108 are fabricated from lubricious materials such as, but not limited to, polyethylene, polypropylene, polytetrafluoroethylene, FEP, materials as described in FIG. 8A, or the like. The inner layer 1102 and the outer layer 1108 can have a thickness ranging from 0.0005 inches to 0.015 inches with a preferred range of 0.001 to 0.010 inches. The elastomeric layer 1104 can range in thickness from 0.001 inches to 0.015 inches with a preferred range of 0.002 to 0.010 inches. In another embodiment, the inner layer 1102, the outer layer 1108, and the elastomeric layer 1104 can advantageously be constructed of clear or transparent polymers to allow for visualization of the tissue surrounding the sheath tube 1100 by means of an endoscope passed through the central lumen 1110. A right angle or side-viewing endoscope, for example 30 degree, 70 degree or 90 degree off axis viewing scope, enhances the ability for viewing out the side of the sheath tube 1100. At minimum, a view is available through the spaces between the coil windings or braid of the reinforcing layer 1106. The reinforcing layer 1106 is as described FIG. 6A. Furthermore, the reinforcing layer 1106 can be fabricated from transparent polymers to further enhance visibility. This construction is beneficial for both the proximal non-expandable region and the distal expandable region of the sheath. In an embodiment, the C-Flex thermoplastic elastomer is used for the elastomeric layer 1104 because it fuses well to the polyethylene exterior layer 1108. This embodiment provides for improved kink resistance, improved bendability, and reduced roughness or bumpiness on the surface of the sheath where the elastomeric layer 1104 shields the reinforcing layer 1106. This embodiment provides for a very smooth surface, which is beneficial on both the interior and exterior surfaces of the sheath. In another embodiment, the sheath tube 1100 is perforated with side holes or fenestrations. These fenestrations are typically smaller in diameter than the diameter of the sheath tube 1100. The fenestrations (not shown) operably connect the internal lumen 1110 to the environment outside the sheath tube 1100. These fenestrations can be round, oval, square, or any other geometric shape.

The present invention may be embodied in other specific forms without departing from its spirit or essential characteristics. For example, the sheath may include instruments affixed integrally to the sheath, rather than being separately inserted, for performing therapeutic or diagnostic functions. Lubricious coatings other than those described may be used and those coatings may be placed on the sheath, the dilator/obturator, or both. The hub may comprise tie downs or configuration changes to permit attachment the hub to the skin of the patient. The hub may further be internally fluted to match the inner lumen fluting of the sheath. The embodiments described herein further are suitable for fabricating sheaths suitable for urological or other transluminal access. The described embodiments are to be considered in all respects only as illustrative and not restrictive. The scope of the invention is therefore indicated by the appended claims rather than the foregoing description. All changes that come within the meaning and range of equivalency of the claims are to be embraced within their scope.

The invention may be embodied in other specific forms without departing from its spirit or essential characteristics. The described embodiments are to be considered in all respects only as illustrative and not restrictive. The scope of the invention is therefore indicated by the appended claims rather than the foregoing description. All changes that come within the meaning and range of equivalency of the claims are to be embraced within their scope.

What is claimed is:

1. A transluminal sheath adapted for insertion into a mammalian body vessel or cavity comprising:
an axially elongate composite sheath tube with a proximal and a distal end and a central through lumen, said composite sheath tube comprising an outer layer, an inner layer, and a reinforcing layer, wherein the outer layer and the inner layer are fabricated from polymeric materials;
a hub coupled to the proximal end of the sheath tube,
a central obturator that is configured to occlude the central lumen of the sheath during insertion; and
a guidewire lumen within the obturator.

2. The transluminal sheath of claim 1, wherein the polymeric material is polyethylene.

3. The transluminal sheath of claim 1, wherein the polymeric material is selected from a blend of high-density and low-density polyethylene.

4. The transluminal sheath of claim 1, wherein the inner and outer layer are fabricated from different polymers.

5. The transluminal sheath of claim 1, wherein the inner or outer layer are fabricated from a blend of high-density and low-density polyethylene wherein the ratio is 25% of one polymer and 75% of the other polymer.

6. The transluminal sheath of claim 1, wherein the sheath bends predominantly by plastic deformation rather than elastic deformation.

7. The transluminal sheath of claim 1, wherein the sheath bends predominantly by plastic deformation, said deformation being enhanced by subjecting the sheath to temperatures substantially near those of body temperature.

8. The transluminal sheath of claim 1, wherein the reinforcing layer is a braided structure fabricated from polymeric materials.

9. The transluminal sheath of claim 1, wherein the reinforcing layer is a braided structure fabricated from polyethylene napthalate.

10. The transluminal sheath of claim 1, wherein the reinforcing layer is a braided structure fabricated from polyethylene terephthalate.

11. The transluminal sheath of claim 1, wherein the reinforcing layer is a braided structure fabricated from a blend of PET and PEN, wherein the ratio of PET to PEN can vary from 0.1 to 0.9.

12. The transluminal sheath of claim 1, wherein the reinforcing layer is a braided structure fabricated from polyamide.

13. The transluminal sheath of claim 1, wherein the reinforcing layer is a braided structure with a pick count ranging between 10 and 30 picks per inch and between 10 and 40 carriers of strand.

14. The transluminal sheath of claim 1, wherein the reinforcing layer is a braided structure comprising fibers with primary diameters between 0.001 inches and 0.010 inches.

15. The transluminal sheath of claim 1, wherein the reinforcing layer is substantially deformable in cross-section.

16. The transluminal sheath of claim 1, wherein the composite sheath tube is substantially deformable in cross-section in response to irregularly shaped objects being advanced or withdrawn therethrough.

17. The transluminal sheath of claim 1, wherein the reinforcement is a coil of flat wire.

18. The transluminal sheath of claim 1, wherein all materials comprising the inner member, the reinforcing member, and the outer member are fabricated from polymeric materials.

19. The transluminal sheath of claim 1, wherein the sheath comprises a radiopaque marker.

20. The transluminal sheath of claim 1, wherein the sheath comprises radiopaque markers substantially near the distal end of the sheath.

21. The transluminal sheath of claim 1 wherein the obturator comprises a radiopaque marker substantially near the distal end of said obturator.

22. The transluminal sheath of claim 1 wherein the reinforcing layer comprises coiled flat wire is spaced at a distance substantially equal to or less than the width of the wire.

23. The transluminal sheath of claim 1, wherein the reinforcing layer comprises flat wire with rounded corners to minimize the risk of polymer wall break-through.

24. The transluminal sheath of claim 1, wherein the reinforcing layer comprises flat wire, which is fabricated from metals.

25. The transluminal sheath of claim 1, wherein the reinforcing layer comprises flat wire which is fabricated from stainless steel.

26. The transluminal sheath of claim 1, wherein the reinforcing layer comprises flat wire is fabricated from polymers.

27. The transluminal sheath of claim 1, wherein the reinforcing layer comprises flat wire is fabricated from PET.

28. The transluminal sheath of claim 1, wherein the reinforcing layer comprises a radiopaque coating.

29. The transluminal sheath of claim 1, wherein the reinforcing layer comprises a radiopaque coating of gold with a thickness of 50 to 500 microns.

30. The transluminal sheath of claim 1, wherein the reinforcing layer, the inner layer, or the outer layer comprise radiopaque materials added into the polymer.

31. The transluminal sheath of claim 1, wherein the reinforcing layer, the inner layer, or the outer layer comprise barium or bismuth compounds added to the polymer during processing.

32. The transluminal sheath of claim 1, wherein the obturator further comprises a tapered tip capable of dilating tissue when the sheath is advanced and of selectively bending through tortuosity over a guidewire.

33. The transluminal sheath of claim 1 further comprising a cap hub on the obturator that securely and reversibly fastens to the hub on the sheath and locks in two substantially orthogonal planes.

34. The transluminal sheath of claim 1 further comprising lubricity enhancing coatings on the outer surface, the inner surface, or both, of the sheath tube.

35. The transluminal sheath of claim 1 further comprising lubricity enhancing coatings on the outer surface of the obturator.

36. The sheath of claim 1 wherein the inner surface of the sheath comprises longitudinally running ridges and adjacent valleys, the presence of such ridges and valleys such that friction between the inner liner and instrumentation, or materials, being passed therethrough, is minimized.

37. The sheath of claim 1, wherein the inner surface of the inner layer of the sheath comprises longitudinally running flutes, the presence of such flutes minimizing the friction between the inner layer and instrumentation or material being advanced or withdrawn therethrough.

38. The sheath of claim 1, wherein the inner surface of the inner layer is dimpled to enhance liquid surface coating of said liner.

39. The sheath of claim 1, wherein the outer surface of the outer layer comprises longitudinally oriented ridges and adjacent valleys, the purpose of which is to enhance liquid transmission along the outer surface of said sheath.

40. The sheath of claim 1 further comprising an atraumatic tip.

41. The sheath of claim 1 comprising distinct regions of increased flexibility with distinct transition regions, moving from the proximal end to the distal end of the sheath.

42. The sheath of claim 1 further comprising drainage holes located along the length of the sheath, said drainage holes cut completely through the sheath wall and communicating between the central lumen of the sheath and the region outside the wall of the sheath.
